(19) United States
(12) Patent Application Publication
(10) Pub. No.: US 2025/0099400 A1
(43) Pub. Date: Mar. 27, 2025

(54) TERPENES FOR USE IN MODULATION OF A PHYSIOLOGICAL FUNCTION

(71) Applicant: BUZZELET DEVELOPMENT AND TECHNOLOGIES LTD., Or Akiva (IL)

(72) Inventors: Noa RAZ, Gizo (IL); Aharon EYAL, Jerusalem (IL)

(21) Appl. No.: 18/931,161

(22) Filed: Oct. 30, 2024

Related U.S. Application Data
(63) Continuation-in-part of application No. PCT/IB2023/054497, filed on Apr. 30, 2023.
(60) Provisional application No. 63/337,111, filed on May 1, 2022.
(60) Provisional application No. 63/614,638, filed on Dec. 25, 2023.

(51) Int. Cl. A61K 31/01; A61K 31/00

(52) U.S. Cl. CPC A61K 31/01; A61K 31/658

(57) ABSTRACT

A composition comprising at least one terpene for use in modulation of a physiological function via at least one selected from the group consisting of agonism of a cannabinoid type 1 receptor (CB1R), modulation of agonism of a CB1R, modulation of antagonism of a CB1R, agonism of a cannabinoid type 2 receptor (CB2R), modulation of agonism of a CB2R, modulation of antagonism of a CB2R and combinations thereof; and methods of use thereof.

### CROSS-REFERENCE TO RELATED APPLICATIONS

This application claims priority from U.S. Provisional Patent Application No. 63/614,638 filed Dec. 25, 2023, and is a Continuation-in-Part of International Application No. PCT/IB2023/054497, which has an international filing date of Apr. 30, 2023, and claims the benefit of priority from U.S. Provisional Patent Application No. 63/337, 111 filed May 1, 2022, which applications are incorporated by reference as if fully set-forth herein.

### FIELD

The present invention relates to the field of medical treatment, and more specifically to use of terpenes for modulation of a physiological function via modulation of activity of a cannabinoid type 1 or a cannabinoid type 2 receptor.

### BACKGROUND

The cannabinoid type 1 (CB₁) receptor is widely distributed in the brain and peripheral organs where it regulates a wide range of cellular functions and metabolism. Activation and deactivation of the activity of a CB1 receptor therefore affects a wide range of physiological functions and conditions in the body of a subject, such as a human subject.

The cannabinoid type 2 (CB₂) receptor is identified peripherally in the circulating immune cells, the spleen and on macrophage-derived cells including osteocytes, osteoclasts and hepatic cells. Unlike the widespread expression of CB₁ receptors in the central nervous system (CNS), the expression of CB₂ receptors, under normal physiological conditions, is restricted to the brainstem and hippocampal pyramidal neurons. However, CB₂ expression is highly inducible on the reactive microglia in the CNS following inflammation or injury.

Activation and deactivation of the activity of a CB₂ receptor therefore affects a wide range of physiological functions and conditions in the body of a subject, such as pain, immune system related function, inflammation, fibrosis, sclerosis, bone structure, autoimmune diseases, cardiovascular function, gastrointestinal function, liver function, kidney function, neurodegenerative, psychiatric, skin disorders, lung function, cancer or addiction in a human subject.

### SUMMARY

According to an aspect of some embodiments of the present invention, there is provided a composition comprising at least one terpene for use in modulation of a physiological function via at least one selected from the group consisting of agonism of a cannabinoid type 1 receptor (CB1R), modulation of agonism of a CB1R, modulation of antagonism of a CB1R and combinations thereof.

According to an aspect of some embodiments of the present invention, there is provided a method of modulating a physiological function in a subject via at least one selected from the group consisting of agonism of a cannabinoid type 1 receptor (CB1R), modulation of agonism of a CB1R, modulation of antagonism of a CB1R and combinations thereof, the method comprising administering to the subject at least one terpene.

According to an aspect of some embodiments of the present invention, there is provided a composition comprising at least one terpene for use in modulation of a physiological function via at least one selected from the group consisting of agonism of a cannabinoid type 2 receptor (CB2R), modulation of agonism of a CB2R, modulation of antagonism of a CB2R and combinations thereof.

According to an aspect of some embodiments of the present invention, there is provided a method of modulating a physiological function in a subject via at least one selected from the group consisting of agonism of a cannabinoid type 2 receptor (CB2R), modulation of agonism of a CB2R, modulation of antagonism of a CB2R and combinations thereof, the method comprising administering to the subject at least one terpene.

### BRIEF DESCRIPTION OF THE DRAWINGS

Some embodiments of the invention are described herein with reference to the accompanying figures. The description, together with the figures, makes apparent to a person having ordinary skill in the art how some embodiments of the invention may be practiced.

In the drawings:

FIG. 1 schematically illustrates the experimental design used to establish the effect of the composition of the present invention on activation of CB1 receptors;

FIGS. 2A-L are dose response curves of CB1 receptor activated GIRK currents by terpenes. Each graph depicts the response to 4 terpene concentrations (each data point represents the mean±SEM from 4-8 oocytes. Responses were normalized to the response evoked by 10 μM THC at the same oocyte;

FIGS. 3A-H are dose response curves of CB1 receptor activated GIRK currents following co-application of THC and terpenes at natural THC/terpenes w/w ratio. Black symbols and lines represent activation of the receptor by THC alone. Red symbols and lines represent activation of the receptor by co-application of THC and terpene. The w/w ratio between THC and terpene was kept 10:1 throughout (each data point represents mean±SEM from 6-14 oocytes). Only terpenes with significant modulatory effect, (i.e., significant main effect of condition, two-way ANOVA, p<0.05) are presented; and

FIGS. 4A-E are dose response curves showing responses obtained for compositions comprising THC plus a terpene mixture as compared to those obtained with THC alone.

FIGS. 5A-P are dose response curves of CB2 receptor activated GIRK currents by terpenes. Each graph depicts the response to 4 terpene concentrations (each data point represents the mean±SEM from 4-8 oocytes. Responses were normalized to the response evoked by 10 μM THC at the same oocyte;

FIGS. 6A-P are dose response curves of CB2 receptor activated GIRK currents following co-application of THC and terpenes at natural THC/terpenes w/w ratio. Black symbols and lines represent activation of the receptor by THC alone. Red symbols and lines represent activation of the receptor by co-application of THC and terpene. The w/w ratio between THC and terpene was kept 10:1 throughout (each data point represents mean±SEM from 6-14 oocytes).

### DETAILED DESCRIPTION

The present invention relates to use of terpenes for modulation of a physiological function related to activation of a cannabinoid type 1 receptor (CB1R).

The present invention further relates to use of terpenes for modulation of a physiological function related to activation of a cannabinoid type 2 receptor (CB2R).

Unless otherwise defined, all technical and scientific terms used herein have the same meaning as commonly understood by one of ordinary skill in the art to which this invention belongs.

The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

As used herein, the term “modulating” with regard to a physiological function refers to changing, controlling, affecting, managing or influencing a type or magnitude of a physiological function, and includes increasing or decreasing a magnitude, a frequency and/or a severity of the physiological function. “Modulating” may be applied to an abnormal physiological function for treating a condition or a symptom thereof arising from such abnormal function, or to a normal healthy physiological function in order to provide a desired change in the function. Modulation of a physiological function may be achieved, for example by modulation of activation of a receptor involved in modulation of the physiological function, such as by increasing or decreasing activation of the receptor by an agonist or antagonist; or by increasing or decreasing availability of THC to the receptor. Alternatively, modulation may be achieved by increasing or decreasing binding of THC to the receptor. Modulation may be additive or synergistic and may involve allosteric or orthosteric interaction of the agonist or antagonist with the receptor.

As used herein, the term “agonist” is intended to encompass both a full and a partial agonist i.e. an agent which fully or partially activates a receptor to which it binds. The agonist or partial agonist may be a natural or synthetic cannabinoid agonist. According to some embodiments, the agonist is a phytocannabinoid or endocannabinoid.

As used herein, the term “treating” includes ameliorating, mitigating, and reducing the instances of a disease or condition, or the symptoms of a disease or condition.

As used herein, the term “administering” includes any mode of administration, such as oral, subcutaneous, sublingual, transmucosal, parenteral, intravenous, intra-arterial, buccal, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a composition comprising at least one terpene for use in modulation of a physiological function via at least one selected from the group consisting of agonism of a cannabinoid type 1 receptor (CB1R), modulation of agonism of a CB1R, modulation of antagonism of a CB1R and combinations thereof.

According to some embodiments, modulation of agonism of the CB1R, and/or modulation of antagonism of the CB1R comprises modulation of an interaction between the CB1R and an agonist or an antagonist of the CB1R.

According to some embodiments, the composition further comprises at least one CB1R agonist or at least one CB1R antagonist, at a total terpene to total agonist or antagonist weight/weight ratio in the range between about 0.05:1 and 1:1, such as about 0.05:1, about 0.1:1, about 0.2:1, about 0.3:1, about 0.4:1, about 0.5:1, about 0.6:1, about 0.7:1, about 0.8:1, about 0.9:1 or about 1:1.

According to some embodiments, the composition is for co-administration with at least one CB1R agonist or at least one CB1R antagonist, at a total terpene to total agonist or antagonist weight/weight ratio in the range between 0.05:1 and 1:1.

According to some embodiments, the agonist comprises tetrahydrocannabinol (THC).

According to some embodiments, the THC is present at a dosage of from about 1 to about 100 mg, such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg.

According to some embodiments, the terpene is for administration to a subject at a dosage resulting in a concentration of up to about 10 μM of said terpene in the plasma and or serum of the subject, such as about 0.5 μM, about 1 μM, about 2 μM, about 3 μM, about 4 μM, about 5 μM, about 6 μM, about 7 μM, about 8 μM, about 9 μM, or about 10 μM.

According to some embodiments, the terpene is selected from the group consisting of alpha pinene, beta pinene, limonene, terpineol, geraniol, myrcene, ocimene, terpinolene, borneol, linalool, sabinene, eucalyptol, bisabolol, beta caryophyllene, humulene, nerolidol and combinations thereof.

According to some embodiments, the terpene is selected from the group consisting of alpha pinene, beta pinene, limonene, terpineol, geraniol, myrcene, ocimene, terpinolene, borneol, linalool, sabinene, eucalyptol and combinations thereof. According to some such embodiments, the terpene is an agonist or partial agonist of the CB1R.

According to some embodiments, the at least one terpene comprises one terpene, two terpenes, three terpenes, four terpenes, five terpenes, six terpene, seven terpenes, eight terpenes or nine terpenes.

According to some embodiments, the agonism or modulation of the CB1 receptor results in increased activation, such as an increase of at least 10%, at least 20%, at least 30%, at least 40%, at least 50%, at least 60%, at least 70%, at least 80%, at least 90%, at least 100%, at least 150%, at least 200%, at least 300% increase in activation as compared to that obtained with THC in the absence of a terpene. According to some embodiments, the agonism or modulation of the CB1 receptor is decreased activation, such as partial activation, partial or total deactivation, such as a deactivation of at least 10%, at least 20%, at least 30%, at least 40%, at least 50%, at least 60%, at least 70%, at least 80%, at least 90%, or 100%.

According to some embodiments, the modulation of a physiological function comprises modulation of a pathological function. According to some such embodiments, the modulation of the physiological function provides treating of a condition relating to the pathological function, or a symptom thereof. Hence according to some embodiments, there is provided a terpene for use in treating a condition related to activation of CB1R receptor.

According to some embodiments, the agonist comprises tetrahydrocannabinol (THC).

According to some embodiments, the at least one terpene is for administration together with tetrahydrocannabinol (THC) at a terpene to THC weight/weight ratio in the range between about 0.05:1 and about 1:1, wherein the modulation of the CB1R receptor is via modulation of an interaction of the THC with the CB1R. According to some embodiments, administration of the terpene together with THC provides a synergistic effect as compared to administration of either the terpene or THC alone.

According to some embodiments, administration of the terpene together with THC provides an additive effect, wherein the combined effect is similar to the sum of the individual effects of the terpene in the absence of THC and THC in the absence of terpene.

According to some such embodiments, the at least one terpene is selected from the group consisting of beta pinene, linalool, limonene, geraniol, ocimene, borneol, terpineol, sabinene and combinations thereof. According to some embodiments, the at least one terpene comprises one terpene, two terpenes, three terpenes, four terpenes, five terpenes, six terpene or seven terpenes.

According to some embodiments, the terpene to THC weight/weight ratio is about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1, or about 1:1. According to some embodiments, the terpene to THC weight/weight ratio is in the range of from about 0.05:1 to about 0.5:1.

According to some embodiments, the THC is provided in a form suitable for sublingual administration.

According to some embodiments, the THC is provided in a form suitable for administration by a route selected from the group consisting of inhalation, oral and transdermal administration.

According to some embodiments, the terpene is for administration together with THC at a dosage of from about 1 to about 100 mg THC, such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg.

According to some embodiments, modulation of the interaction between the CB1R and the agonist or antagonist of the CB1R is selected from the group consisting of an allosteric interaction, an orthosteric interaction, and a combination thereof.

According to some embodiments, the terpene is for co-administration together with THC in a single dosage form.

According to some embodiments, the terpene is for administration in a separate dosage form from said THC, wherein the administration is performed independently, sequentially, simultaneously or concomitantly. According to some such embodiments, the separate dosage forms may be dosage forms configured for administration by different delivery routes, or dosage forms configured for separate administration by a same delivery route.

According to some embodiments, the terpene is for administration within 2 hours of administration of the THC.

According to some embodiments, the terpene is for administration prior to administration of the THC. According to some such embodiments, the terpene is for administration substantially immediately prior to administration of the THC. According to some embodiments, the terpene is for administration about 1 minute, about 5 minutes, about 10 minutes, about 15 minutes, about 20 minutes, about 30 minutes, about 1 hour or about 2 hours prior to administration of the THC.

According to some embodiments, the terpene is for administration subsequent to administration of the THC. According to some embodiments, the terpene is for administration substantially immediately subsequent to administration of the THC. According to some embodiments, the terpene is for administration about 1 minute, about 5 minutes, about 10 minutes, about 15 minutes, about 20 minutes, about 30 minutes, about 1 hour or about 2 hours subsequent to administration of the THC.

According to some embodiments, the THC is provided in a form selected from the group consisting of a solid, a vapor, an aerosol and a liquid.

According to some embodiments, the terpene is provided in a form selected from the group consisting of a solid, a vapor, an aerosol and a liquid.

According to some embodiments, the terpene is for administration together with a CB1R agonist other than tetrahydrocannabinol (THC) at a terpene to CB1R agonist weight/weight ratio in the range between about 0.05:1 and about 1:1, wherein the modulation of activation of the CB1R is via modulation of an interaction of the non-THC CB1R agonist with the CB1R.

According to some embodiments, the physiological function is selected from the group consisting of pain, sleep, appetite, anxiety, depression, memory, movement, inflammation, excitatory neuronal activity, neurodegeneration, neurotransmitter release, stress, cardiovascular function, motivation, mood, sedation, cognitive function, muscle tension, cramps and combinations thereof.

According to an aspect of some embodiments of the present invention, there is provided a method of modulating a physiological function in a subject via at least one selected from the group consisting of agonism of a cannabinoid type 1 receptor (CB1R); modulation of agonism of a CB1R; modulation of antagonism of a CB1R; and combinations thereof, the method comprising administering to the subject at least one terpene.

According to some embodiments, modulation of agonism of the CB1R, and/or modulation of antagonism of the CB1R, comprises modulation of an interaction between the CB1R and an agonist or an antagonist of the CB1R.

According to some embodiments, the method further comprises administration of at least one CB1R agonist or at least one CB1R antagonist, at a total terpene to total agonist or antagonist weight/weight ratio in the range between about 0.05:1 and 1:1, such as about 0.05:1, about 0.1:1, about 0.2:1, about 0.3:1, about 0.4:1, about 0.5:1, about 0.6:1, about 0.7:1, about 0.8:1, about 0.9:1 or about 1:1.

According to some embodiments, the agonist comprises tetrahydrocannabinol (THC).

According to some embodiments of the method, the THC is administered at a dosage of from about 1 to about 100 mg THC, such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg.

According to some embodiments of the method, the terpene is administered at a dosage resulting in a concentration of up to about 10 μM of said terpene in the plasma and or serum of said subject, such as about 0.5 μM, about 1 μM, about 2 μM, about 3 μM, about 4 μM, about 5 μM, about 6 μM, about 7 μM, about 8 μM, about 9 μM, or about 10 μM.

According to some such embodiments, the at least one terpene comprises one terpene, two terpenes, three terpenes, four terpenes, five terpenes, six terpene or seven terpenes.

According to some embodiments of the method, the terpene is selected from the group consisting of beta pinene, linalool, limonene, geraniol, ocimene, borneol, terpineol, sabinene and combinations thereof.

According to some embodiments, the terpene alpha pinene, beta pinene, limonene, terpineol, geraniol, myrcene, ocimene, terpinolene, borneol, linalool, sabinene, eucalyptol and combinations thereof. According to some such embodiments, the modulation of the CB1 receptor comprises an agonistic or antagonistic interaction with the receptor.

According to some embodiments of the method, the modulation of the CB1 receptor results in increased activation, such as an increase of at least 10%, at least 20%, at least 30%, at least 40%, at least 50%, at least 60%, at least 70%, at least 80%, at least 90%, at least 100%, at least 150%, at least 200%, at least 300% increase in activation as compared to that obtained with THC in the absence of a terpene. According to some embodiments, the modulation of the CB1 receptor results in decreased activation, such as partial or total deactivation, such as a deactivation of at least 10%, at least 20%, at least 30%, at least 40%, at least 50%, at least 60%, at least 70%, at least 80%, at least 90%, or 100%.

According to some embodiments of the method, the modulation of a physiological function comprises modulation of a pathological function. According to some such embodiments, the modulation of the physiological function provides treating of a condition relating to the pathological function, or a symptom thereof. Hence according to some embodiments, there is provided a terpene for use in treating a condition related to activation of a cannabinoid type 1 receptor (CB1R).

According to some embodiments, the method further comprises administering tetrahydrocannabinol (THC) at a terpene to THC weight/weight ratio in the range between 0.05:1 and 1:1, wherein modulation of the CB1 receptor is via modulation of an interaction of the THC with the CB1 receptor. According to some such embodiments, administration of the terpene together with THC provides a synergistic effect as compared to administration of either the terpene or THC alone. According to some such embodiments of the method, the terpene is selected from the group consisting of Beta Pinene, Linalool, Limonene, Geraniol, Ocimene, Borneol, Terpineol, Sabinene and combinations thereof.

According to some embodiments of the method, the terpene to THC weight/weight ratio is about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1, or about 1:1. According to some embodiments, the terpene to THC weight/weight ratio is in the range of from about 0.05:1 to about 0.5:1.

According to some embodiments of the method, the THC is administered sublingually.

According to some embodiments of the method, modulation of the interaction between the CB1R and the agonist or antagonist of the CB1R is selected from the group consisting of an allosteric modulation, an orthosteric modulation and a combination thereof.

According to some embodiments of the method, the terpene and the THC are administered in a single dosage form.

According to some embodiments of the method, the terpene and the THC are administered in separate dosage forms, wherein the administration is performed independently, sequentially, simultaneously or concomitantly. According to some such embodiments, the separate dosage forms are administered by different delivery routes, or in dosage forms by a same delivery route.

According to some embodiments of the method, the terpene is administered within 2 hours of administration of the THC.

According to some embodiments of the method, the terpene is administered prior to administration of the THC. According to some such embodiments, the terpene is administered substantially immediately prior to administration of the THC. According to some embodiments, the terpene is administered about 1 minute, about 5 minutes, about 10 minutes, about 15 minutes, about 20 minutes, about 30 minutes, about 1 hour or about 2 hours prior to administration of the THC.

According to some embodiments of the method, the terpene is administered subsequent to administration of the THC. According to some such embodiments, the terpene is administered substantially immediately subsequent to administration of the THC. According to some embodiments, the terpene is administered about 1 minute, about 5 minutes, about 10 minutes, about 15 minutes, about 20 minutes, about 30 minutes, about 1 hour or about 2 hours subsequent to administration of the THC.

According to some embodiments of the method, the THC is administered in the form of a solid, a vapor, an aerosol or a liquid form.

According to some embodiments of the method, the terpene is administered in a form selected from the group consisting of a solid, a vapor, an aerosol and a liquid.

According to some embodiments, the method further comprises administering a CB1 agonist other than Tetrahydrocannabinol (THC) at a terpene to CB1 agonist weight/weight ratio in the range between about 0.05:1 and about 1:1, wherein the modulation of activation of the CB1 receptor is via modulation of an interaction of the THC with the CB1 receptor.

According to some embodiments of the method, the physiological function is selected from the group consisting of pain, sleep, appetite, anxiety, depression, memory, movement, inflammation, excitatory neuronal activity, neurodegeneration, neurotransmitter release, stress, cardiovascular function, motivation, mood, sedation, cognitive function, muscle tension, cramps and combinations thereof.

According to an aspect of some embodiments of the present invention, there is provided a composition comprising at least one terpene for use in modulation of a physiological function via at least one selected from the group consisting of agonism of a cannabinoid type 2 receptor (CB2R), modulation of agonism of a CB2R, modulation of antagonism of a CB2R and combinations thereof.

According to some such embodiments, the at least one terpene is selected from the group consisting of beta caryophyllene, bisabolol, humulene, nerolidol, limonene, terpineol, geraniol, myrcene, ocimene, terpinolene, borneol, linalool, sabinene, eucalyptol and combinations thereof. According to some embodiments, the at least one terpene comprises one terpene, two terpenes, three terpenes, four terpenes, five terpenes, six terpene, seven terpenes, eight terpenes, nine terpenes, ten terpenes, eleven terpenes, twelve terpenes, thirteen terpenes of fourteen terpenes.

According to some embodiments, modulation of agonism of the CB2R and/or modulation of antagonism of the CB2R comprises modulation of an interaction between the CB2R and an agonist or an antagonist of the CB2R.

According to some embodiments, the composition further comprises at least one CB2R agonist or at least one CB2R antagonist, at a total terpene to total agonist or antagonist weight/weight ratio in the range between about 0.05:1 and 1:1, such as about 0.05:1, about 0.1:1, about 0.2:1, about 0.3:1, about 0.4:1, about 0.5:1, about 0.6:1, about 0.7:1, about 0.8:1, about 0.9:1 or about 1:1.

According to some embodiments, the composition is for co-administration with at least one CB2R agonist or at least one CB2R antagonist, at a total terpene to total agonist or antagonist weight/weight ratio in the range between 0.05:1 and 1:1.

According to some such embodiments, the terpene for co-administration is selected from the group consisting of alpha pinene, beta pinene, linalool, limonene, bisabolol, beta caryophyllene, ocimene, borneol and combinations thereof. According to some embodiments, the at least one terpene comprises one terpene, two terpenes, three terpenes, four terpenes, five terpenes, six terpene, seven terpenes or eight terpenes. According to some such embodiments, the terpene is an agonist or partial agonist of the CB1R.

According to some embodiments, the agonist is selected from the group consisting of cannabidiol (CBD), cannabidiolic acid (CBDA), tetrahydrocannabinol (THC), tetrahydrocannabinolic acid (THCA), cannabidivarin (CBDV), cannabidivarinic acid (CBDVA), tetrahydrocannabidivarin (THCV), tetrahydrocannabidivarinic acid (THCVA), cannabinol (CBN), cannabinolic acid (CBNA), cannabigerol (CBG), cannabigerolic acid (CBGA) and combinations thereof.

According to some embodiments, the agonist is present at a dosage of from about 1 to about 100 mg, such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg.

According to a preferred embodiment, the agonist comprises tetrahydrocannabinol (THC).

According to some embodiments, the THC is present at a dosage of from about 1 to about 100 mg, such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, 95 mg or about 100 mg.

According to some embodiments, the terpene is for administration to a subject at a dosage resulting in a concentration of up to about 10 μM of said terpene in the plasma and or serum of the subject, such as about 0.5 μM, about 1 μM, about 2 μM, about 3 μM, about 4 μM, about 5 μM, about 6 μM, about 7 μM, about 8 μM, about 9 μM, or about 10 μM.

According to some embodiments, the agonism or modulation of the CB2 receptor results in increased activation, such as an increase of at least 10%, at least 20%, at least 30%, at least 40%, at least 50%, at least 60%, at least 70%, at least 80%, at least 90%, at least 100%, at least 150%, at least 200%, at least 300% increase in activation as compared to that obtained with THC in the absence of a terpene. According to some embodiments, the agonism or modulation of the CB2 receptor is decreased activation, such as partial activation, partial or total deactivation, such as a deactivation of at least 10%, at least 20%, at least 30%, at least 40%, at least 50%, at least 60%, at least 70%, at least 80%, at least 90%, or 100%.

According to some embodiments, the modulation of a physiological function comprises modulation of a pathological function. According to some such embodiments, the modulation of the physiological function provides treating of a condition relating to the pathological function, or a symptom thereof. Hence according to some embodiments, there is provided a terpene for use in treating a condition related to activation of CB2R receptor.

According to some embodiments, the agonist is selected from the group consisting of cannabidiol (CBD), cannabidiolic acid (CBDA), tetrahydrocannabinol (THC), tetrahydrocannabinolic acid (THCA), cannabidivarin (CBDV), cannabidivarinic acid (CBDVA), tetrahydrocannabidivarin (THCV), tetrahydrocannabidivarinic acid (THCVA), cannabinol (CBN), cannabinolic acid (CBNA), cannabigerol (CBG), cannabigerolic acid (CBGA) and combinations thereof. According to a preferred embodiment, the agonist comprises tetrahydrocannabinol (THC).

According to some embodiments, the at least one terpene is for administration together with tetrahydrocannabinol (THC) at a terpene to THC weight/weight ratio in the range between about 0.05:1 and about 1:1, wherein the modulation of the CB2R receptor is via modulation of an interaction of the THC with the CB2R. According to some embodiments, administration of the terpene together with THC provides a synergistic effect as compared to administration of either the terpene or THC alone.

According to some embodiments, administration of the terpene together with THC provides an additive effect, wherein the combined effect is similar to the sum of the individual effects of the terpene in the absence of THC and THC in the absence of terpene.

According to some embodiments, the terpene to THC weight/weight ratio is about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1, or about 1:1. According to some embodiments, the terpene to THC weight/weight ratio is in the range of from about 0.05:1 to about 0.5:1.

According to some embodiments, the THC is provided in a form suitable for sublingual, oral, buccal, nasal, dermal, transdermal, or inhaled administration.

According to some embodiments, the terpene is for administration together with THC at a dosage of from about 1 to about 100 mg THC, such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg.

According to some embodiments, modulation of the interaction between the CB2R and the agonist or antagonist of the CB2R is selected from the group consisting of an allosteric interaction, an orthosteric interaction, and a combination thereof.

According to some embodiments, the terpene is for co-administration together with THC in a single dosage form.

According to some embodiments, the terpene is for administration in a separate dosage form from said THC, wherein the administration is performed independently, sequentially, simultaneously or concomitantly. According to some such embodiments, the separate dosage forms may be dosage forms configured for administration by different delivery routes, or dosage forms configured for separate administration by a same delivery route.

According to some embodiments, the terpene is for administration within 2 hours of administration of the THC.

According to some embodiments, the terpene is for administration prior to administration of the THC. According to some such embodiments, the terpene is for administration substantially immediately prior to administration of the THC. According to some embodiments, the terpene is for administration about 1 minute, about 5 minutes, about 10 minutes, about 15 minutes, about 20 minutes, about 30 minutes, about 1 hour or about 2 hours prior to administration of the THC.

According to some embodiments, the terpene is for administration subsequent to administration of the THC. According to some embodiments, the terpene is for administration substantially immediately subsequent to administration of the THC. According to some embodiments, the terpene is for administration about 1 minute, about 5 minutes, about 10 minutes, about 15 minutes, about 20 minutes, about 30 minutes, about 1 hour or about 2 hours subsequent to administration of the THC.

According to some embodiments, the THC is provided in a form selected from the group consisting of a solid, a vapor, an aerosol and a liquid.

According to some embodiments, the terpene is provided in a form selected from the group consisting of a solid, a vapor, an aerosol and a liquid.

According to some embodiments, the terpene is for administration together with a CB2R agonist other than tetrahydrocannabinol (THC) at a terpene to CB2R agonist weight/weight ratio in the range between about 0.05:1 and about 1:1, wherein the modulation of activation of the CB2R is via modulation of an interaction of the non-THC CB2R agonist with the CB2R.

According to some embodiments, the physiological function is selected from the group consisting of pain, immune system related function, inflammation, fibrosis, sclerosis, bone structure, autoimmune diseases, cardiovascular function, gastrointestinal function, liver function, kidney function, neurodegenerative, psychiatric, skin disorders, lung function, cancer, addiction and combinations thereof in a human subject.

According to an aspect of some embodiments of the present invention, there is provided a method of modulating a physiological function in a subject via at least one selected from the group consisting of agonism of a cannabinoid type 2 receptor (CB2R); modulation of agonism of a CB2R; modulation of antagonism of a CB2R; and combinations thereof, the method comprising administering to the subject at least one terpene.

According to some such embodiments, the at least one terpene is selected from the group consisting of beta caryophyllene, bisabolol, humulene, nerolidol, limonene, terpineol, geraniol, myrcene, ocimene, terpinolene, borneol, linalool, sabinene, eucalyptol and combinations thereof. According to some embodiments, the at least one terpene comprises one terpene, two terpenes, three terpenes, four terpenes, five terpenes, six terpene, seven terpenes, eight terpenes, nine terpenes, ten terpenes, eleven terpenes, twelve terpenes, thirteen terpenes of fourteen terpenes.

According to some embodiments, modulation of agonism of the CB2R, and/or modulation of antagonism of the CB2R, comprises modulation of an interaction between the CB2R and an agonist or an antagonist of the CB2R.

According to some embodiments, the method further comprises administration of at least one CB2R agonist or at least one CB2R antagonist, at a total terpene to total agonist or antagonist weight/weight ratio in the range between about 0.05:1 and 1:1, such as about 0.05:1, about 0.1:1, about 0.2:1, about 0.3:1, about 0.4:1, about 0.5:1, about 0.6:1, about 0.7:1, about 0.8:1, about 0.9:1 or about 1:1. According to some such embodiments, the terpene for co-administration is selected from the group consisting of alpha pinene, beta pinene, linalool, limonene, bisabolol, beta caryophyllene, ocimene, borneol and combinations thereof. According to some embodiments, the at least one terpene comprises one terpene, two terpenes, three terpenes, four terpenes, five terpenes, six terpene, seven terpenes or eight terpenes.

According to some embodiments, the agonist is selected from the group consisting of cannabidiol (CBD), cannabidiolic acid (CBDA), tetrahydrocannabinol (THC), tetrahydrocannabinolic acid (THCA), cannabidivarin (CBDV), cannabidivarinic acid (CBDVA), tetrahydrocannabidivarin (THCV), tetrahydrocannabidivarinic acid (THCVA), cannabinol (CBN), cannabinolic acid (CBNA), cannabigerol (CBG), cannabigerolic acid (CBGA) and combinations thereof.

According to some embodiments, the agonist is present at a dosage of from about 1 to about 100 mg, such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg.

According to a embodiment, the agonist comprises preferred tetrahydrocannabinol (THC).

According to some embodiments of the method, the THC is administered at a dosage of from about 1 to about 100 mg THC, such as about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg.

According to some embodiments of the method, the terpene is administered at a dosage resulting in a concentration of up to about 10 μM of said terpene in the plasma and or serum of said subject, such as about 0.5 μM, about 1 μM, about 2 μM, about 3 μM, about 4 μM, about 5 μM, about 6 μM, about 7 μM, about 8 μM, about 9 μM, or about 10 μM.

According to some such embodiments, the at least one terpene comprises one terpene, two terpenes, three terpenes, four terpenes, five terpenes, six terpene or seven terpenes.

According to some embodiments of the method, the terpene is selected from the group consisting of alpha pinene, beta pinene, linalool, limonene, bisabolol, beta caryophyllene, ocimene, borneol and combinations thereof.

According to some embodiments, the terpene is selected from the group consisting of beta caryophyllene, bisabolol, humulene, nerolidol, limonene, terpineol, geraniol, myrcene, ocimene, terpinolene, borneol, linalool, sabinene, eucalyptol and combinations thereof. According to some such embodiments, the modulation of the CB2 receptor comprises an agonistic or antagonistic interaction with the receptor.

According to some embodiments of the method, the modulation of the CB2 receptor results in increased activation, such as an increase of at least 10%, at least 20%, at least 30%, at least 40%, at least 50%, at least 60%, at least 70%, at least 80%, at least 90%, at least 100%, at least 150%, at least 200%, at least 300% increase in activation as compared to that obtained with THC in the absence of a terpene. According to some embodiments, the modulation of the CB2 receptor results in decreased activation, such as partial or total deactivation, such as a deactivation of at least 10%, at least 20%, at least 30%, at least 40%, at least 50%, at least 60%, at least 70%, at least 80%, at least 90%, or 100%.

According to some embodiments of the method, the modulation of a physiological function comprises modulation of a pathological function. According to some such embodiments, the modulation of the physiological function provides treating of a condition relating to the pathological function, or a symptom thereof. Hence according to some embodiments, there is provided a terpene for use in treating a condition related to activation of a cannabinoid type 2 receptor (CB2R).

According to some embodiments, the method further comprises administering tetrahydrocannabinol (THC) at a terpene to THC weight/weight ratio in the range between 0.05:1 and 1:1, wherein modulation of the CB2 receptor is via modulation of an interaction of the THC with the CB2 receptor. According to some such embodiments, administration of the terpene together with THC provides a synergistic effect as compared to administration of either the terpene or THC alone. According to some such embodiments of the method, the terpene is selected from the group consisting of alpha pinene, beta pinene, linalool, limonene, bisabolol, beta caryophyllene, ocimene, borneol and combinations thereof.

According to some embodiments of the method, the terpene to THC weight/weight ratio is about 0.05:1, about 0.06:1, about 0.07:1, about 0.08:1, about 0.09:1, about 0.1:1, about 0.2:1, about 0.3:1, about 0.4:1, about 0.5:1, about 0.6:1, about 0.7:1, about 0.8:1, about 0.9:1, or about 1:1. According to some embodiments, the terpene to THC weight/weight ratio is in the range of from about 0.05:1 to about 0.5:1.

According to some embodiments of the method, the THC is administered sublingually.

According to some embodiments of the method, modulation of the interaction between the CB2R and the agonist or antagonist of the CB2R is selected from the group consisting of an allosteric modulation, an orthosteric modulation and a combination thereof.

According to some embodiments of the method, the terpene and the THC are administered in a single dosage form.

According to some embodiments of the method, the terpene and the THC are administered in separate dosage forms, wherein the administration is performed independently, sequentially, simultaneously or concomitantly. According to some such embodiments, the separate dosage forms are administered by different delivery routes, or in dosage forms by a same delivery route.

According to some embodiments of the method, the terpene is administered within 2 hours of administration of the THC.

According to some embodiments of the method, the terpene is administered prior to administration of the THC. According to some such embodiments, the terpene is administered substantially immediately prior to administration of the THC. According to some embodiments, the terpene is administered about 1 minute, about 5 minutes, about 10 minutes, about 15 minutes, about 20 minutes, about 30 minutes, about 1 hour or about 2 hours prior to administration of the THC.

According to some embodiments of the method, the terpene is administered subsequent to administration of the THC. According to some such embodiments, the terpene is administered substantially immediately subsequent to administration of the THC. According to some embodiments, the terpene is administered about 1 minute, about 5 minutes, about 10 minutes, about 15 minutes, about 20 minutes, about 30 minutes, about 1 hour or about 2 hours subsequent to administration of the THC.

According to some embodiments of the method, the THC is administered in the form of a solid, a vapor, an aerosol or a liquid form.

According to some embodiments of the method, the terpene is administered in a form selected from the group consisting of a solid, a vapor, an aerosol and a liquid.

According to some embodiments, the method further comprises administering a CB2 agonist other than Tetrahydrocannabinol (THC) at a terpene to CB2 agonist weight/weight ratio in the range between about 0.05:1 and about 1:1, wherein the modulation of activation of the CB2 receptor is via modulation of an interaction of the THC with the CB2 receptor.

According to some embodiments of the method, the physiological function is selected from the group consisting of pain, immune system related function, inflammation, fibrosis, sclerosis, bone structure, autoimmune diseases, cardiovascular function, gastrointestinal function, liver function, kidney function, neurodegenerative, psychiatric, skin disorders, lung function, cancer, addiction and combinations thereof in a human subject.

### EXAMPLES

### Materials and Methods

### Materials

All chemicals were purchased from Sigma (Rehovot, Israel) unless stated otherwise.

THC was extracted from a THC-rich cannabis chemovar, using an authorized IGMP (Israeli Good Manufactory Practice) extraction process at Bazelet manufacturing plant (Or Akiva, Israel) and verified by a validated High Performance Liquid Chromatography (HPLC) analysis (HPLC Waters PDA 2996, equipped with a pump, autosampler, column-oven, and a Photodiode Array detector (PDA) detector).

Purified terpenes were purchased from Vigon International Inc. (Pennsylvania, USA. α-pinene (natural, 98.2%), β-pinene (natural, 94%) limonene-D (natural, 99%), myrcene (natural, 95.5%), ocimene (Trans, natural, 69.3%) sabinene (natural, 76.67%), terpinolene (natural, 92.6%), borneol (natural, 59.9%), eucalyptol (natural, 100%), geraniol natural (97%), linalool (racemic mixture, 100%), terpineol (natural, 98%), β-caryophyllene (natural, 88.4%), humulene (natural, 91.6%), bisabolol (natural, 98.5%) and nerolidol (natural, 99%).

Stock solutions were prepared containing 10 mM THC or 10 mM terpene (on pure basis) in DMSO, based on compound individual purity levels. Subsequent dilutions were made in 24 mM K⁺ solution (see above). Similarly, a 10 mM stock solution in DMSO was prepared from 2-Arachidonoylglycerol (2-AG), purchased from Sigma (Rehovot, Israel).
Preparation of cRNA and Oocytes.

cDNA plasmids of the two subunits of the G protein-activated inwardly rectifying K⁺ channel (GIRK) (GIRK1 and GIRK2), the CB1 receptor, the CB2 receptor, and the α subunit of the G-protein (Gαi3) were linearized with the appropriate restriction enzymes. The linearized plasmids were transcribed in vitro using a standard procedure.

Oocytes were isolated from female adult X. laevis anesthetized (with 1 g/L MS-222) and incubated in NDE96 solution composed of ND96 (in mM: 96 NaCl, 2 KCl, 1 CaCl₂, 1 MgCl₂, 5 Hepes, with pH adjusted to 7.5 with NaOH) with the addition of 2.5 mM Na⁺ pyruvate, 100 units/ml penicillin, and 100 μg/ml streptomycin (16). The isolated oocytes were injected with the relevant cRNAs: cRNAs of CB1 receptor and CB2 receptor (2 ng) and GIRK1 and GIRK2 (200 pg each) were injected. In addition, cRNA of Gαi3 (1000 pg) was injected to decrease the basal GIRK current (I_{K}) and to improve the relative activation by the agonist.

### Current Measurements

Currents were measured 3-5 days after cRNA injection and were recorded using the standard two-electrode voltage clamp technique (Axoclamp 2B amplifier, Axon Instruments, Foster City, CA).

Each oocyte was placed in a recording bath containing ND96 solution and was impaled with two electrodes pulled from 1.5-mm Clark capillaries (Warner instruments, Hamden, CT). Both electrodes were filled with a 3M KCI solution and the electrode resistance was between 1 and 5 MΩ. The CB1 receptor-mediated GIRK and the CB2 receptor-mediated GIRK currents were measured in 24 mM K⁺ solution (in mM: 72 NaCl, 24 KCl, 1 CaCl2, 1 MgCl2, 5 Hepes, with pH adjusted to 7.5 with KOH). Ba⁺² (1 mM) was used to block the currents in order to verify that the measured currents were indeed mediated by GIRK channels.

pCLAMP10 software (Axon Instruments, Molecular Devices, San Jose, CA) was used for data acquisition and analysis.

### Data Analysis

The dose response curves were fitted by equation 1:

Y=Bottom+X*(Top-Bottom)/(EC₅₀+X), (1)

where Y is the normalized response, X is the concentration of THC, Bottom and Top are the lowest and highest points of the curve and EC₅₀ is the THC concentration that gives the half-maximal response. For all experiments, 10 μM was taken as the highest THC concentration, as dictated by solubility limit. Therefore, at the end of the recording from each oocyte, the response to 10 μM was measured as a reference value and all recordings were normalized to this value.

The responses evoked in the same oocyte by either THC and\or terpenes measurements, were normalized to this reference value. Given that the dose-response curves did not reach saturation, the THC concentration that evoked 50% of the response evoked by 10 μM THC was defined as the apparent EC₅₀.

### Statistical Analysis

Statistical analysis was conducted using Statistical SPSS 20.0 software (IBM Corp., Armonk, N.Y.). One and Two-way Analysis of Variance (ANOVA) tests were used to evaluate the effects of terpenes on CB1 responses, and the effects of terpenes on the THC-derived CB1 responses. Post-hoc tests using Bonferroni adjustment for multiple comparisons were conducted to detect differences between groups.

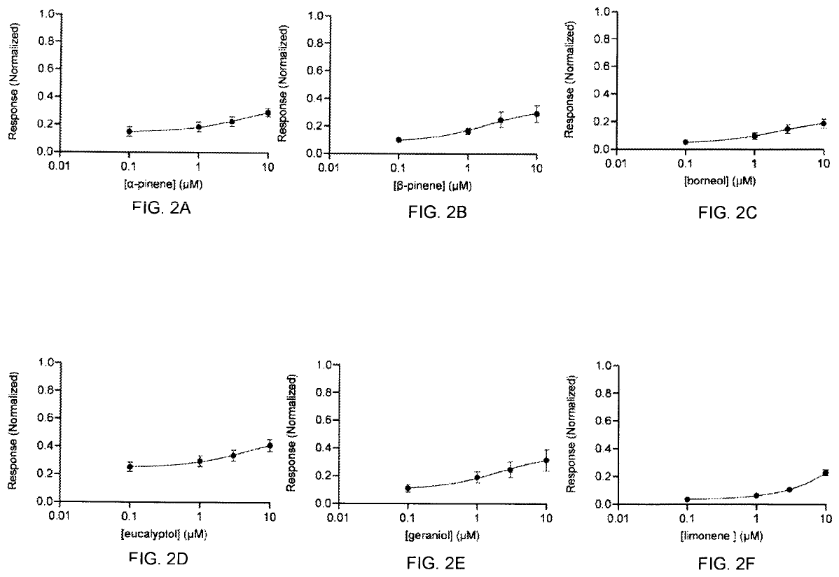

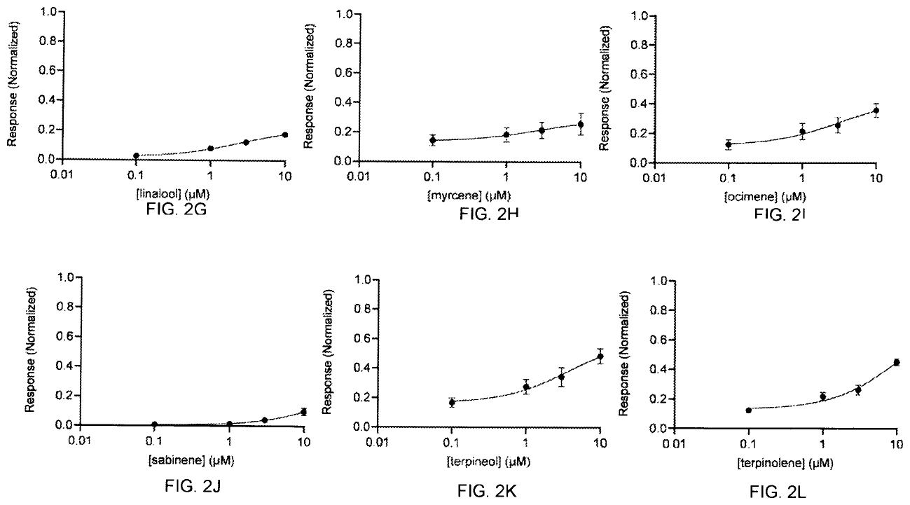

A one-way ANOVA was used to evaluate dose-dependent CB1 activity of the various terpenes (FIG. 2).

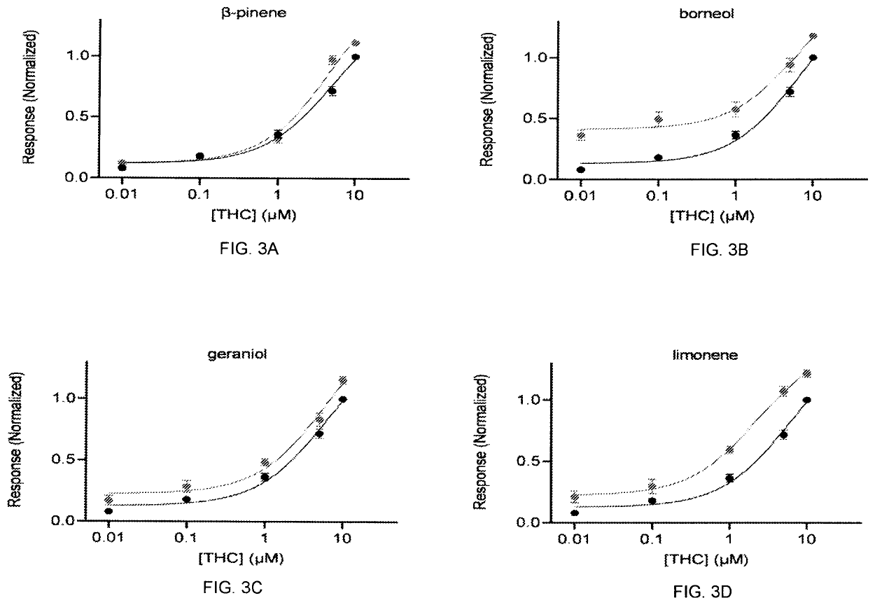

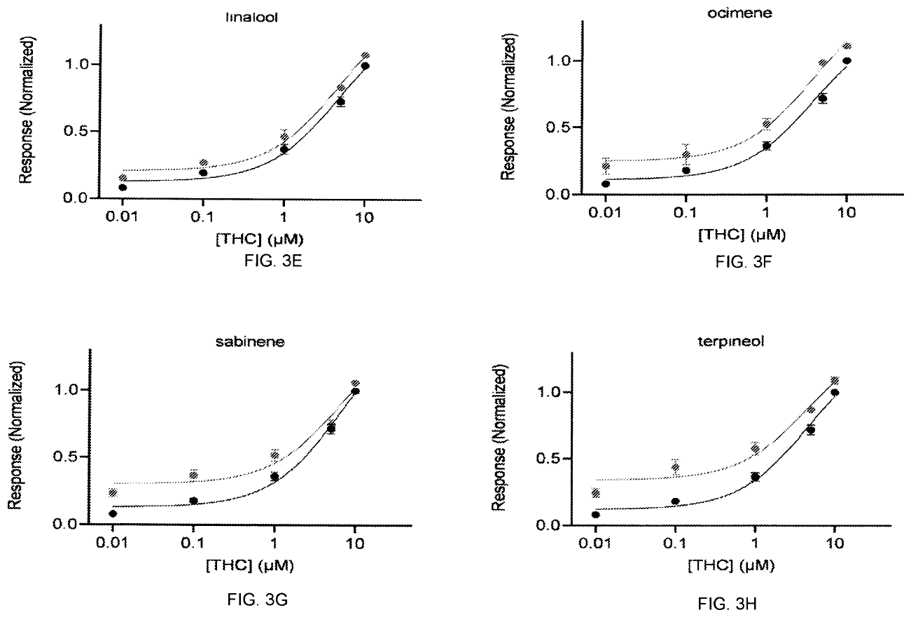

Two-way ANOVA was used to evaluate terpenes effects on THC-derived CB1 responses (FIG. 3), analyzing the two main effects of (1) Condition, i.e., the CB1 response obtained by application of THC alone vs. the response obtained by co-application of THC and a terpene, and (2) THC concentration levels (0.01, 0.1, 1 and 5 μM THC). An interaction between the main effects was also assessed.

Data for the 10 μM THC was excluded from the analysis since this data point was used for normalization at each measurement (defined as 1).

### Example 1: Effect of Terpenes on CB1 Receptor Activation

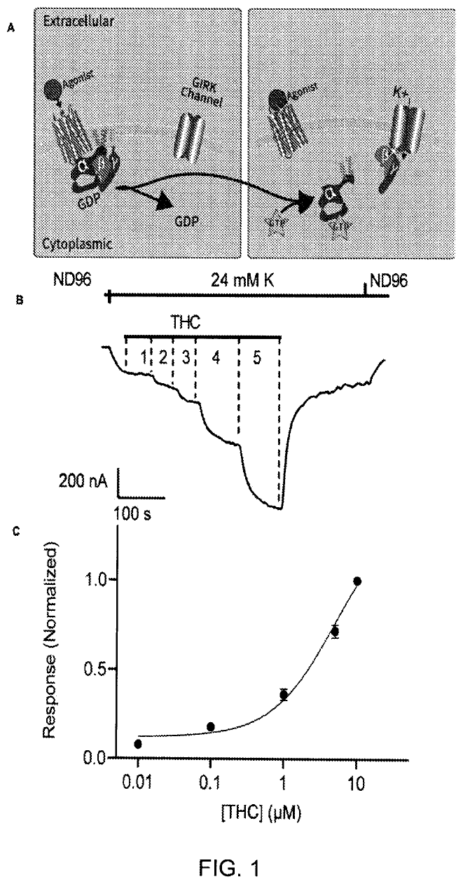

The Xenopus oocytes functional expression system was used to test the possibility that the presence of various terpenes affects the activation of the CB1 receptor, as depicted schematically in FIG. 1A. To this end, Xenopus oocytes were injected with cRNAs of proteins involved in the pathway leading to activation of K⁺ currents by CB1 receptor via βγ subunits of the G-proteins: The CB1 receptor, the two subunits of the GIRK channel (GIRK1 and GIRK2), and the Gαi3 subunit.

First, the dependence of THC-induced K⁺ current (I_{THC}) on THC concentration (dose-response, DR) was measured. The oocyte was voltage-clamped to −80 mV in a low K⁺ (2 mM K⁺) solution, ND96. Basal GIRK current (I_{K}) was developed upon replacement of the ND96 by the 24 mM K⁺solution. This current represents the basal activation of GIRK channel by endogenous By subunits present in the oocytes. Then, five different concentrations of THC were applied sequentially in ascending order giving rise to I_{THC}. This current was terminated upon washout of THC. Employing this basic experimental protocol, a full DR curve was constructed. A representative recording is shown in FIG. 1B.

In order to compare between oocytes, I_{THC} at any particular THC concentration was normalized to I_{THC} obtained at a 10 μM THC, defined as the reference response, at this same oocyte.

The effects of terpenes on CB1 receptor activation and on THC-induced CB1 receptor activation were next assessed. Sixteen cannabis terpenes were studied, including: α-pinene, β-pinene, limonene, myrcene, ocimene, sabinene and terpinolene (monoterpenes, hydrocarbons consisting of two isoprene units, having the molecular formula of C10H16), borneol, eucalyptol, geraniol, linalool and terpineol, (monoterpenoids, oxygen-containing monoterpenes, C10H18O), β-caryophyllene and humulene (sesquiterpenes, hydrocarbons consisting of three isoprene units, C15H24), bisabolol and nerolidol (sesquiterpenoids, oxygen-containing sesquiterpenes, C15H260).

Terpene-derived CB1 activations are presented in FIG. 1, depicting DR curves of the various terpenes. Twelve out of the 16 terpenes were tested (β-caryophyllene, bisabolol, humulene and nerolidol were excluded from analysis as their solubility is below the tested concentration range). The responses to each of the terpene in each experiment was normalized to the response evoked by 10 μM THC in the same oocyte, taken to be 1. As seen, CB1 receptor activity is detected for all terpenes. The response to 10 μM terpene ranged between 10% and 48% of the response amplitude obtained by the reference 10 μM THC.

To study terpene effects on the THC-activated CB1 receptor response, the CB1 receptor activation by THC alone was compared to its activation by the same THC concentrations, each supplemented with terpene at a weight/weight ratio of 1/10. The results demonstrate that the addition of beta pinene, borneol, geraniol, limonene, linalool, ocimene, sabinene and terpineol at this ratio significantly enhances the potency of THC in CB1 receptor activation.

### Example 2: Effect of THC Plus Terpene Mixtures on CB1 Receptor Activation

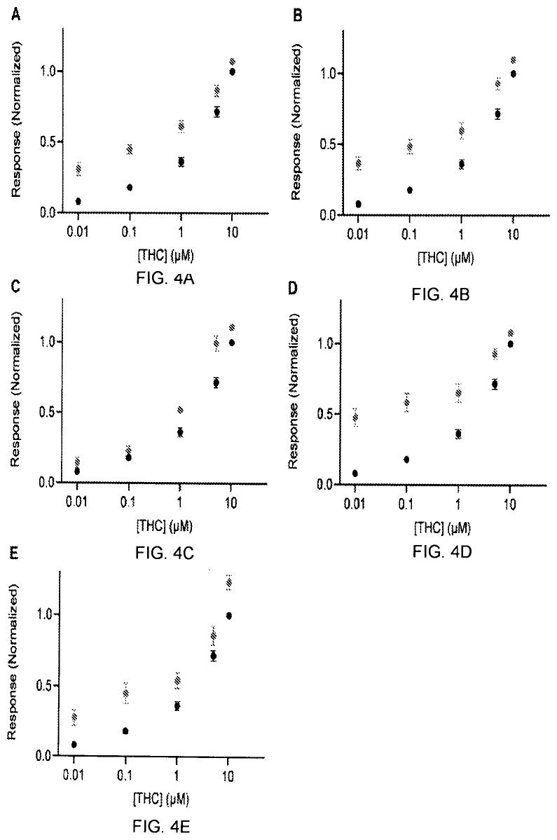

Compositions A-E were prepared comprising THC and a terpene mixture as specified below, wherein the concentration ratio of THC to total terpene mixture was 1:10:
A=THC, alpha pinene, sabinene, limonene, terpinolene, eucalyptol, borneol, beta caryophyllene
B=THC, myrcene, ocimene, linalool, terpineol, beta caryophyllene, nerolidol, bisabolol
C=THC, alpha pinene, beta pinene, eucalyptol, linalool, terpineol, borneol, beta caryophyllene, nerolidol
D=THC, beta pinene, myrcene, terpineol, borneol, beta caryophyllene, nerolidol
E=THC, alpha pinene, beta pinene, limonene, terpinolene, geraniol, beta caryophyllene, bisabolol
   The effect of each composition on activation of the CB1 receptor was tested as detailed above for Example 1. THC alone was used as a control.
   Results are presented in FIGS. 4A-E, showing that a greater response was obtained for a composition comprising THC plus a terpene mixture as compared to that obtained with THC alone.
   Example 3: Effects of Terpenes on CB2 receptor activation

The effects of terpenes on CB₂ receptor activation and on THC-induced CB₂ receptor activation were assessed. Sixteen cannabis terpenes were studied, including: α- pinene, β-pinene, limonene, myrcene, ocimene, sabinene and terpinolene (monoterpenes, hydrocarbons consisting of two isoprene units, having the molecular formula of C10H16), borneol, eucalyptol, geraniol, linalool and terpineol, (monoterpenoids, oxygen-containing monoterpenes, C10H18O), β-caryophyllene and humulene (sesquiterpenes, hydrocarbons consisting of three isoprene units, C15H24), bisabolol and nerolidol (sesquiterpenoids, oxygen-containing sesquiterpenes, C15H26O).

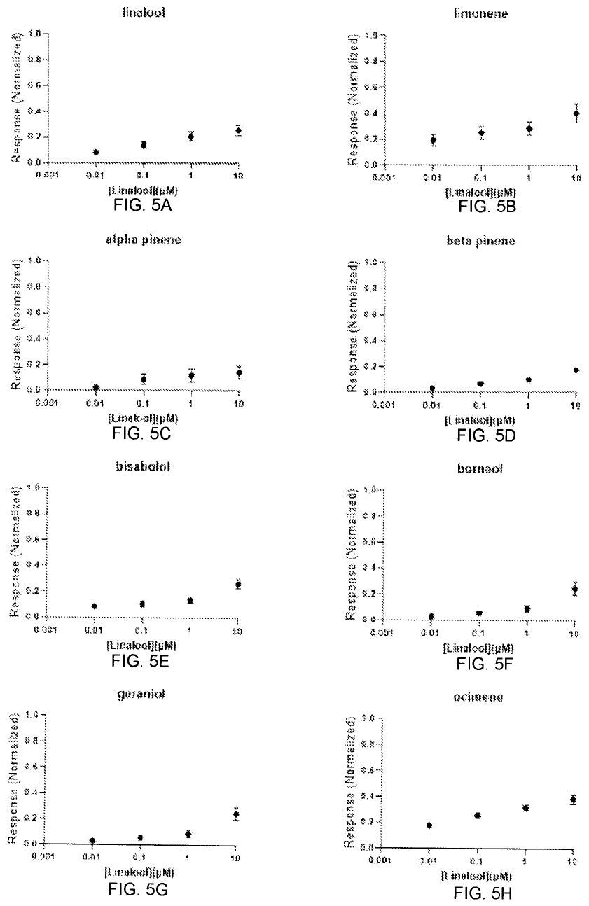

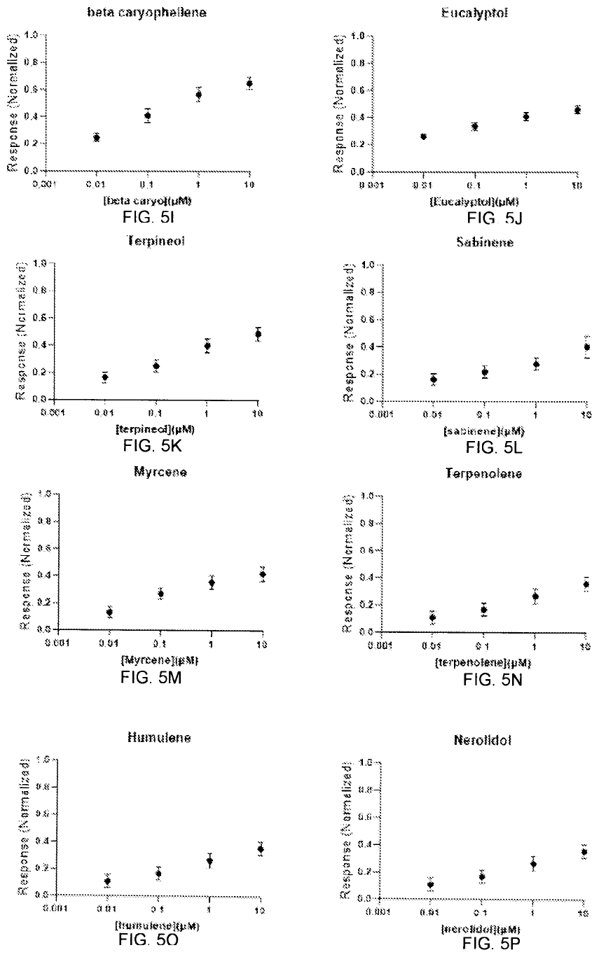

Terpene-derived CB₂ activations are presented in FIGS. 5A-P (in order from left to right and top to bottom), depicting DR curves of the various terpenes. The responses to each of the terpene in each experiment was normalized to the response evoked by 10 μM THC in the same oocyte, taken to be 1. As seen, CB₂ receptor activity is detected for all terpenes. The response to 10 μM terpene ranged between 15% and 65% of the response amplitude obtained by the reference 10 μM THC.

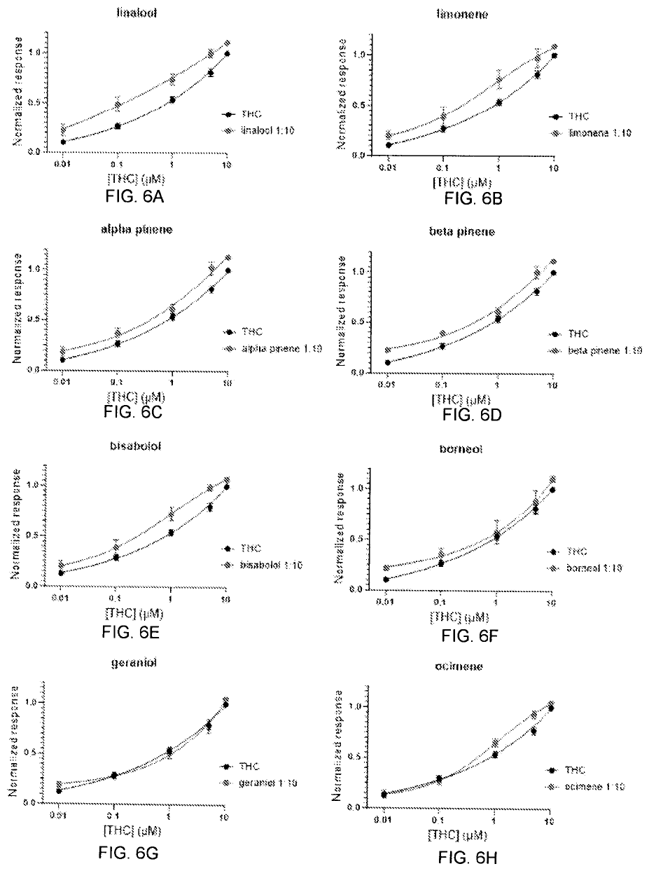

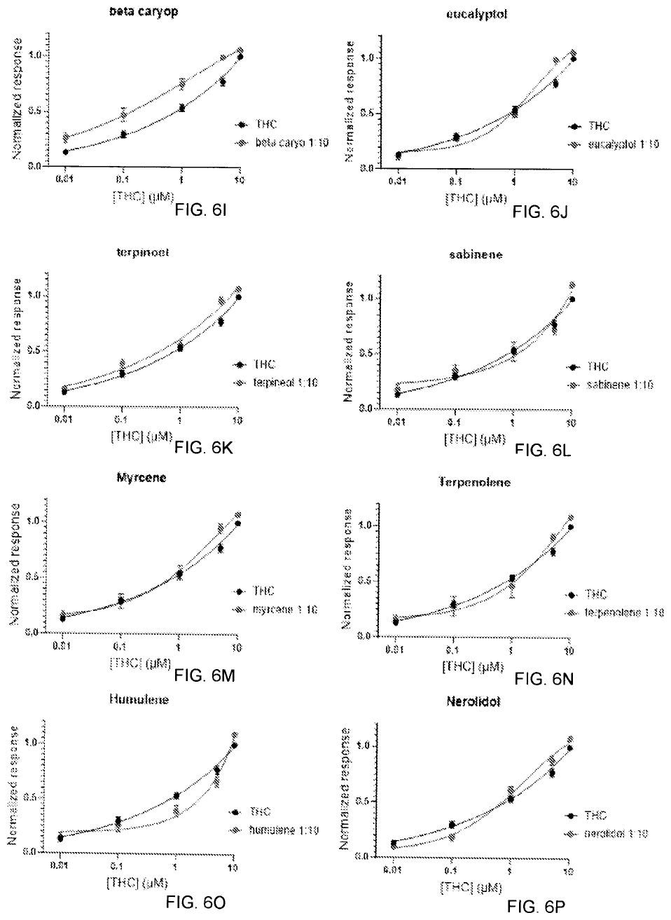

To study terpene effects on the THC-activated CB2 receptor response, the CB2 receptor activation by THC alone was compared to its activation by the same THC concentrations, each supplemented with terpene at a weight/weight ratio of 1/10. Results are presented in FIGS. 6A-3P (in order from left to right and top to bottom).

Statistical analysis was made based on the method disclosed in Raz et al (Biochem. Pharamacol. 2023), which is incorporated by reference as if fully set out herein.

What is claimed is:

1. A method of modulating a physiological function in a subject via at least one selected from the group consisting of agonism of a cannabinoid type 1 receptor (CB1R), modulation of agonism of a CB1R, modulation of antagonism of a CB1R, agonism of a cannabinoid type 2 receptor (CB2R), modulation of agonism of a CB2R, modulation of antagonism of a CB2R and combinations thereof, the method comprising administering to the subject at least one terpene.

2. The method of claim 1, wherein said modulation comprises modulation of an interaction between said CB1R and an agonist or an antagonist of said CB1R.

3. The method of claim 1, further comprising administering at least one CB1R agonist or at least one CB1R antagonist, at a total terpene to total agonist or antagonist weight/weight ratio in the range between 0.05:1 and 1:1.

4. The method of claim 2, wherein said agonist comprises tetrahydrocannabinol (THC).

5. The method of claim 4, wherein said THC is administered at a dosage of from about 1 to about 100 mg THC.

6. The method of claim 1, wherein said terpene is selected from the group consisting of alpha pinene, beta pinene, limonene, terpineol, geraniol, myrcene, ocimene, terpinolene, borneol, linalool, sabinene, eucalyptol, bisabolol, beta caryophyllene, humulene, nerolidol and combinations thereof.

7. The method of claim 1, wherein said terpene is selected from the group consisting of alpha pinene, beta pinene, limonene, terpineol, geraniol, myrcene, ocimene, terpinolene, borneol, linalool, sabinene, eucalyptol and combinations thereof.

8. The method of claim 1, wherein said terpene is selected from the group consisting of alpha pinene, beta pinene, linalool, limonene, bisabolol, beta caryophyllene, ocimene, borneol and combinations thereof.

9. The method of claim 1, wherein said terpene is selected from the group consisting of beta caryophyllene, bisabolol, humulene, nerolidol, limonene, terpineol, geraniol, myrcene, ocimene, terpinolene, borneol, linalool, sabinene, eucalyptol and combinations thereof.

10. The method of claim 4, wherein said terpene and said THC are administered in a single dosage form.

11. The method of claim 4, wherein said terpene and said THC are administered in separate dosage forms.

12. The method of claim 2, wherein said modulation of said interaction between said CB1R and said agonist or said antagonist of said CB1R is selected from the group consisting of an allosteric modulation, an orthosteric modulation and a combination thereof.

13. The method of claim 1, wherein said modulation is via at least one selected from the group consisting of agonism of a cannabinoid type 1 receptor (CB1R), modulation of agonism of a CB1R, modulation of antagonism of a CB1R and said physiological function is selected from the group consisting of pain, sleep, appetite, anxiety, depression, memory, movement, inflammation, excitatory neuronal activity, neurodegeneration, neurotransmitter release, stress, cardiovascular function, motivation, mood, sedation, cognitive function, muscle tension, cramps and combinations thereof.

14. The method of claim 1, wherein said modulation comprises modulation of an interaction between said CB2R and an agonist or an antagonist of said CB2R.

15. The method of claim 14, further comprising administering at least one CB2R agonist or at least one CB2R antagonist, at a total terpene to total agonist or antagonist weight/weight ratio in the range between 0.05:1 and 1:1.

16. The method of claim 15, wherein said agonist comprises tetrahydrocannabinol (THC).

17. The method of claim 16, wherein said THC is administered at a dosage of from about 1 to about 100 mg THC.

18. The method of claim 15, wherein said terpene and said THC are administered in a single dosage form.

19. The method of claim 15, wherein said terpene and said THC are administered in separate dosage forms.

20. The method of claim 1, wherein said modulation is via at least one selected from the group consisting of agonism of a cannabinoid type 2 receptor (CB2R), modulation of agonism of a CB2R, modulation of antagonism of a CB2R and combinations thereof, and said physiological function is selected from the group consisting of pain, immune system related function, inflammation, fibrosis, sclerosis, bone structure, autoimmune diseases, cardiovascular function, gastrointestinal function, liver function, kidney function, neurodegenerative, psychiatric, skin disorders, lung function, cancer, addiction and combinations thereof.
